(19) United States
(12) Patent Application Publication
(10) Pub. No.: US 2023/0287322 A1
(43) Pub. Date: Sep. 14, 2023

(54) METHODS AND SYSTEMS FOR PRODUCING POLYPEPTIDES

(71) Applicant: Seagen Inc., Bothell, WA (US)

(72) Inventors: Matthew John LEITH, Bothell, WA (US); Cheng-Wei Aaron CHEN, Bothell, WA (US); John Devachariam BUSHEY, Bothell, WA (US); Swapnil BHARGAVA, Bothell, WA (US)

(21) Appl. No.: 18/016,648

(22) PCT Filed: Jul. 27, 2021
(86) PCT No.: PCT/US2021/070985

§ 371 Date: Jan. 17, 2023

Related U.S. Application Data
(60) Provisional application No. 63/057,800, filed on Jul. 28, 2020.

(51) Int. Cl. C12M 1/00; C12P 21/02; B01D 61/14; B01D 69/02

(52) U.S. Cl. CPC C12M 29/04; C12P 21/02; C12M 47/10; C12M 29/10; B01D 61/149; B01D 69/02; B01D 2315/10; B01D 2311/04; B01D 61/147; B01D 61/145; B01D 2325/02834; B01D 2325/34

(57) ABSTRACT

Provided herein are methods and systems for production (e.g., batch production) of a polypeptide product via cell culture. In some embodiments, the methods and systems use a first bioreactor (e.g., for cell culturing), an alternating tangential flow (ATF) microfilter (e.g., for removing a polypeptide product and culture medium from the cell culture while retaining cells), a second bioreactor (e.g., for concentrating the product), and an ATF ultrafilter (e.g., for retaining product in the second bioreactor and allowing culture medium to exit).

### CROSS REFERENCE TO RELATED APPLICATIONS

This application claims the priority benefit of U.S. Provisional Application Ser. No. 63/057,800, filed Jul. 28, 2020, which is incorporated herein by reference in its entirety.

### FIELD

The present disclosure relates to methods and systems for producing polypeptide product(s) from a cell culture.

### BACKGROUND

Production and purification of polypeptide products (e.g., monoclonal antibodies and antibody fragments, among other biologics) on a production scale is extremely valuable for both research and pharmaceutical manufacturing. However, production of polypeptides on such a scale presents problems due to the complex nature of cell culture-based production and downstream purification/concentration processes. While using continuous operation for process intensification has been readily applied to chemical manufacturing, discontinuous production methods such as fed-batch culturing have been favored for production of polypeptide products. However, it can be challenging to attain very high product concentrations using fed-batch culturing methods because product can only accumulate so much within the cell cultures.

Some methods related to continuous cell culture processes (e.g through use of perfusion techniques) have been developed. See, e.g., US20170114381, EP1720972A1, WO2018022661, and WO2005095578. However, process intensification through the use of perfusion at the production stage makes the purification process extremely difficult due to the need for a continuous harvest and purification. Another well-recognized challenge of the perfusion process is the need to clarify a massive quantity of cells.

The most commonly used practice with process intensification to bridge the gap between cell culture and the first polypeptide capture stage is to utilize a break tank and continuous chromatography over an extended period of time (Konstantinov, K. B. and Cooney, C. L. (2015). J. Pharma. Sci. 104:P813-820). The challenges with this approach are related to quality and regulatory concerns, in that it becomes difficult to define a lot/batch of material in regards to particular specifications.

As such, there remains a need for methods of polypeptide production through cell culturing that allow for improved process intensification and compatibility with downstream purification steps.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

Provided herein are methods and systems for production (e.g., batch production) of a polypeptide product. In some embodiments, these methods and systems use a harvest vessel separate from the production bioreactor, along with micro- and ultra-filters specifically configured with the production bioreactor and harvest vessel to provide enhanced process intensification. The present disclosure demonstrates that the methods and systems described herein allow product to be concentrated during production for extended periods of time prior to any downstream purification, providing greater product concentration, while also optionally retaining use of a single (or limited number of) batch(es) per production process. This is thought to allow for retaining downstream operations used in standard batch production and/or reducing costs (e.g., eliminating the need for downstream depth filtration, centrifugation, or other methods to clarify large numbers of cells).

In some aspects, provided herein are methods for producing a polypeptide. In some embodiments, the methods comprise: (a) culturing, in a culture medium in a first bioreactor (e.g., a production bioreactor), a host cell that expresses the polypeptide under conditions suitable for expression of the polypeptide, wherein the first bioreactor is in fluid connection with an alternating tangential flow (ATF) microfilter such that the host cell, the culture medium, and the polypeptide from the first bioreactor contact the ATF microfilter; (b) transferring the polypeptide and a portion of the culture medium through the ATF microfilter into a second bioreactor (e.g., a harvest vessel or bioreactor) that is in fluid connection with the ATF microfilter, wherein the ATF microfilter causes the host cell to be retained in the first bioreactor and allows the polypeptide and the portion of the culture medium to pass into the second bioreactor; (c) contacting the polypeptide and the portion of the culture medium in the second bioreactor with an ATF ultrafilter that is in fluid connection with the second bioreactor, wherein the ATF ultrafilter causes the polypeptide to be retained in the second bioreactor and allows culture medium to exit the second bioreactor; and (d) collecting the polypeptide from the second bioreactor.

In some embodiments according to any of the embodiments described herein, the polypeptide is collected from the second bioreactor in one or more non-continuous batches. In some embodiments, the host cell is cultured in the first bioreactor for a period, and the polypeptide is collected from the second bioreactor in one batch per period. In some embodiments, the host cell is cultured in the first bioreactor for a period of about 2 weeks to about 3 weeks, about 2 weeks to about 4 weeks, about 2 weeks to about 8 weeks, up to about 8 weeks, up to about 12 weeks or longer than about 12 weeks, and the polypeptide is collected from the second bioreactor in one batch per period. In some embodiments, the host cell is cultured in the first bioreactor for a period of about 14 days to about 21 days, about 14 days to about 30 days, about 14 days to about 60 days, up to about 60 days, up to about 90 days or longer than about 90 days, and the polypeptide is collected from the second bioreactor in one batch per period. In some embodiments, the host cell is cultured in the first bioreactor for a period, and the polypeptide is collected from the second bioreactor in more than one batch per period. In some embodiments, the host cell is cultured in the first bioreactor for a period of more than about 3 weeks, more than about 4 weeks, more than about 8 weeks, more than about 12 weeks, or more than about 18 weeks, and the polypeptide is collected from the second bioreactor in more than one batch per period. In some embodiments, the host cell is cultured in the first bioreactor for a period of more than about 21 days, more than about 30 days, more than about 60 days, more than about 90 days, or more than about 120 days, and the polypeptide is collected from the second bioreactor in more than one batch per period. In some embodiments, the polypeptide is collected at a concentration of at least about 1 g/L, at least about 5 g/L, at least about 7 g/L, at least about 8 g/L, or at least about 10 g/L. In some embodiments, the polypeptide is collected at a concentration of about 3 g/L to about 5 g/L, about 3 g/L to about 8 g/L, about 3 g/L to about 10 g/L, about 5 g/L to about 8 g/L, or about 5 g/L to about 10 g/L.

In some embodiments according to any of the embodiments described herein, culturing the host cell in the first bioreactor and/or transferring the polypeptide to the second bioreactor are performed in a continuous manner. In some embodiments, culturing the host cell in the first bioreactor and transferring the polypeptide to the second bioreactor are both performed in a continuous manner. In some embodiments, the polypeptide is collected from the second bioreactor in a non-continuous manner. In some embodiments, culturing the host cell in the first bioreactor and transferring the polypeptide to the second bioreactor are both performed in a continuous manner, and the polypeptide is collected from the second bioreactor in a non-continuous manner. In some embodiments, culturing the host cell in the first bioreactor and transferring the polypeptide to the second bioreactor are performed simultaneously. In some embodiments, culturing the host cell in the first bioreactor and/or transferring the polypeptide to the second bioreactor are performed more than once prior to contacting the polypeptide and the portion of the culture medium in the second bioreactor with the ATF ultrafilter and collecting the polypeptide from the second bioreactor. In some embodiments, culturing the host cell in the first bioreactor and transferring the polypeptide to the second bioreactor are both performed more than once prior to contacting the polypeptide and the portion of the culture medium in the second bioreactor with the ATF ultrafilter and collecting the polypeptide from the second bioreactor. In some embodiments, the methods further comprise, prior to contacting the polypeptide and the portion of the culture medium in the second bioreactor with the ATF ultrafilter and collecting the polypeptide from the second bioreactor, culturing the host cell in a culture medium in the first bioreactor (e.g., a production bioreactor) and transferring the polypeptide and a second portion of the culture medium through the ATF microfilter into the second bioreactor. In some embodiments, the polypeptide and the portion of the culture medium in the second bioreactor are contacted with the ATF ultrafilter more than once prior to collecting the polypeptide from the second bioreactor. In some embodiments, the polypeptide is transferred to the second bioreactor more than once (e.g., in two or more batches) prior to collecting the polypeptide from the second bioreactor. In some embodiments, the methods further comprise, prior to contacting the polypeptide and the portion of the culture medium in the second bioreactor with the ATF ultrafilter, removing a second portion of the culture medium from the second bioreactor through the ATF ultrafilter. In some embodiments, the second portion of the culture medium is less than the first portion. In some embodiments, the second portion of the culture medium is removed from the second bioreactor when volume of culture medium in the second bioreactor reaches a predetermined volume. In some embodiments, concentration of the polypeptide in the second bioreactor after removing the second portion is greater than concentration of the polypeptide in the second bioreactor prior to removing the second portion. In some embodiments, the polypeptide is collected from the second bioreactor when concentration of the polypeptide in the second bioreactor reaches a predetermined concentration, e.g., about 1 g/L, about 3 g/L, about 5 g/L, about 8 g/L, or about 10 g/L. In some embodiments, the methods further comprise (e.g., prior to collecting the polypeptide from the second bioreactor and/or while the host cell is cultured in the first bioreactor) introducing additional culture medium into the first bioreactor. In some embodiments, additional culture medium is introduced into the first bioreactor at a rate that is approximately equivalent to a rate of transferring the portion of the culture medium from the first bioreactor into the second bioreactor (e.g., in (b)). In some embodiments, the host cell is cultured in a perfusion cell culture (e.g., in (a)). In some embodiments, the methods further comprise (e.g., prior to collecting the polypeptide from the second bioreactor and/or while the host cell is cultured in the first bioreactor) introducing additional culture medium into the first bioreactor at a rate of about 1 volume of the first bioreactor per day. In some embodiments, the portion of the culture medium is transferred from the first bioreactor to the second bioreactor at a rate of about 1 volume of the first bioreactor per day (e.g., prior to collecting the polypeptide from the second bioreactor). In some embodiments, the methods further comprise (e.g., after collecting the polypeptide from the second bioreactor): purifying the collected polypeptide via one or more downstream purification processes. In some embodiments, the one or more downstream purification processes do not include or comprise depth filtration. In some embodiments, the methods further comprise (e.g., after collecting the polypeptide from the second bioreactor): contacting the collected polypeptide with protein A. In some embodiments, the methods further comprise (e.g., after collecting the polypeptide from the second bioreactor): subjecting the collected polypeptide to protein A affinity chromatography.

In some embodiments according to any of the embodiments described herein, the ATF microfilter has a pore size of about 750 kD to about 0.4 μm. In some embodiments, the ATF microfilter has a pore size of about 0.2 μm. In some embodiments, the ATF microfilter has a pore size that is smaller than the host cell and larger than the polypeptide. In some embodiments, the ATF ultrafilter has a molecular weight cutoff of about 30 kD to about 100 kD. In some embodiments, the ATF ultrafilter has a molecular weight cutoff of about 30 kD to about 50 kD. In some embodiments, the ATF ultrafilter has a molecular weight cutoff that is less than a molecular weight of the polypeptide.

In some embodiments according to any of the embodiments described herein, the polypeptide is a secreted polypeptide. In some embodiments, the polypeptide is a monoclonal antibody or antibody fragment (e.g., an antigen-binding fragment of a monoclonal antibody). In some embodiments, the host cell is a mammalian host cell. In some embodiments, the host cell is a Chinese hamster ovary (CHO) cell. In some embodiments, the culture medium is a defined culture medium.

In other aspects, provided herein are systems for production (e.g., batch production) of a polypeptide. In some embodiments, the systems comprise: a first bioreactor; an alternating tangential flow (ATF) microfilter; a second bioreactor; and an ATF ultrafilter. In some embodiments, the first bioreactor is in fluid connection with the ATF microfilter. In some embodiments, the ATF microfilter is in fluid connection with the first bioreactor and the second bioreactor, and the ATF microfilter causes cells to be retained in the first bioreactor and allows culture medium and the polypeptide to pass into the second bioreactor. In some embodiments, the second bioreactor is in fluid connection with the ATF microfilter and the ATF ultrafilter, and the ATF ultrafilter causes the polypeptide to be retained in the second bioreactor. In some embodiments, the ATF ultrafilter allows culture medium to exit the second bioreactor.

In some embodiments according to any of the embodiments described herein, the first bioreactor is a stirred tank bioreactor. In some embodiments, the first bioreactor is a stirred tank bioreactor with a volume of about 3 L to about 3000 L. In some embodiments, the first bioreactor is a 3 L stirred tank bioreactor. In some embodiments, the second bioreactor is a stirred tank bioreactor. In some embodiments, the second bioreactor is a stirred tank bioreactor with a volume of about 3 L to about 3000 L. In some embodiments, the second bioreactor is a 3 L stirred tank bioreactor. In some embodiments, the systems further comprise a permeate pump connected to the ATF microfilter and the second bioreactor. In some embodiments, the permeate pump causes culture medium and the polypeptide to pass through the ATF microfilter into the second bioreactor. In some embodiments, the systems further comprise a permeate pump connected to the second bioreactor and the ATF ultrafilter. In some embodiments, the permeate pump causes culture medium to exit the second bioreactor through the ATF ultrafilter. In some embodiments, the systems further comprise a first permeate pump connected to the ATF microfilter and the second bioreactor, and a second permeate pump connected to the second bioreactor and the ATF ultrafilter. In some embodiments, the first permeate pump causes culture medium and the polypeptide to pass through the ATF microfilter into the second bioreactor, and the second permeate pump causes culture medium to exit the second bioreactor through the ATF ultrafilter. In some embodiments, the permeate pump connected to the second bioreactor and the ATF ultrafilter is configured to operate when a predetermined volume is reached in the second bioreactor. In some embodiments, the predetermined volume is between 100 mL and 5000 L. In some embodiments, the predetermined volume is 1.5 L. In some embodiments, the systems further comprise a waste outlet or waste collection vessel connected to the ATF ultrafilter. In some embodiments, the waste outlet or collection vessel is configured to remove or retain culture medium from the second bioreactor through the ATF ultrafilter.

In some embodiments according to any of the embodiments described herein, the ATF microfilter has a pore size of about 750 kD to about 0.4 μm. In some embodiments, the ATF microfilter has a pore size of about 0.2 μm. In some embodiments, the ATF microfilter has a pore size that is smaller than the host cell and larger than the polypeptide. In some embodiments, the ATF ultrafilter has a molecular weight cutoff of about 30 kD to about 100 kD. In some embodiments, the ATF ultrafilter has a molecular weight cutoff of about 30 kD to about 50 kD. In some embodiments, the ATF ultrafilter has a molecular weight cutoff that is less than a molecular weight of the polypeptide.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 provides a schematic diagram of an exemplary system for batch production of a polypeptide, in accordance with some embodiments. FIG. 1 illustrates the use of the exemplary system, utilizing two Alternating Tangential Flow (ATF) filters attached to the production and harvest vessels. FIG. 1 shows that the Production Bioreactor retains the cells but allows for constant removal of the polypeptide to the Harvest Vessel, where the polypeptide is then concentrated using an ultrafilter.

FIG. 2 shows the titer (mg/L) of an exemplary polypeptide product measured over time (days) in the production bioreactor (e.g., a first bioreactor as described herein) and the harvest vessel (e.g., a second bioreactor as described herein) of an exemplary system for batch production of a polypeptide, in accordance with some embodiments. FIG. 2 illustrates a constant polypeptide production rate in the first bioreactor and a subsequent polypeptide concentration in the second bioreactor. The last two data points illustrate a further concentration of the second bioreactor prior to downstream processing.

### DETAILED DESCRIPTION

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

As used herein, a “culture medium” includes any nutrient solution used to support a cell culture (e.g., a mammalian cell culture, such as a CHO cell culture). Generally, a culture medium provides amino acids (e.g., one or more essential amino acids, or all amino acids), an energy source (e.g., a sugar such as glucose), vitamins and other organic compounds, trace elements (e.g., inorganic compounds or elements required at very low concentrations), and lipids. Culture media can include media comprising serum as well as defined or serum-free media. In some embodiments, the culture medium is a perfusion culture medium. In some embodiments, a culture medium is supplemented with one or more additional components that supports or enhances the growth and/or health of a cell culture, including for example hormones or growth factors (e.g., insulin, serum, transferrin, epidermal or other growth factors, etc.), buffers, salts, nucleobases, protein digests or hydrolysates (e.g., peptones or plant or animal hydrolysates), anti-apoptotic compounds, antibiotics, antimycotics, and surfactants (e.g., non-ionic surfactants such as block co-polymers, polyethylene glycols, or polyvinyl alcohols).

As used herein, a “bioreactor” refers to any vessel or apparatus used for cell culture (e.g., mammalian cell culture, such as culturing CHO cells). A bioreactor may be suitable for use in any stage of cell culturing, including without limitation inoculation, expansion, and production bioreactors. Examples of bioreactors include, without limitation, stirred tank, wave, centrifugal, multi-stage, hollow fiber, fluidized bed, fermentor type, immobilized cell, air lift type, and packed bed bioreactors.

“Continuous” (when used in reference to cell culture or culturing herein) may refer to cell culturing in which a product (e.g., a polypeptide produced by the cell culture, such as an antibody) and portions of culture medium are removed from a bioreactor (e.g., a bioreactor containing the cell culture, such as a production bioreactor described herein) continuously during cell culturing.

“Non-continuous” (when used in reference to cell culture or culturing herein) may refer to cell culturing in which a product (e.g., a polypeptide produced by the cell culture, such as an antibody) is removed from a bioreactor (e.g., a bioreactor containing the product and at least a portion of culture medium, such as a harvest vessel described herein) in one or more distinct or discontinuous batches.

A “perfusion” cell culture may refer to a cell culture that is maintained by introducing fresh culture medium and removing spent culture medium during the cell culture, e.g., continuously. In some embodiments, a perfusion cell culture comprises a separation or filtration method/apparatus to retain cells in the culture while removing culture medium or permeate.

As used herein, “antibody” broadly encompasses monoclonal antibodies (including full length antibodies comprising an immunoglobulin Fc region), single-chain molecules, multispecific antibodies (e.g., bispecific antibodies, diabodies, trispecific antibodies, etc.), as well as antigen-binding antibody fragments thereof. For example, native antibodies are heterotetrameric glycoproteins of about 150 kD composed of two light chains and two heavy chains. Each light chain is linked to a heavy chain by 1 covalent disulfide bond, while the number of disulfide linkages varies among the heavy chains of different immunoglobulin isotypes (e.g., IgA, IgD, IgE, IgG, and IgM, including the subtypes IgG1, IgG2, IgG3, IgG4, IgA1, and IgA2). Each heavy and light chain also has regularly spaced intra-chain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

Antibodies typically comprise a constant domain (e.g., the portion of an immunoglobulin molecule having a more conserved amino acid sequence relative to the variable domain, and including the CH1, CH2 and CH3 domains of the heavy chain and the CL domain of the light chain) and a variable domain that contains the antigen binding site (typically at the N-terminal ends of the heavy and light chains). Within each variable domain (e.g., both heavy and light chain variable domains, abbreviated as the VH and VL domains respectively) are three segments called complementarity-determining regions (CDRs) interspersed with 4 more highly conserved portions of the variable domains known as framework regions (FR). Each VH or VL domain typically comprises 4 FR regions, largely adopting a beta-sheet configuration, connected by 3 CDRs, which form loops connecting, and in some cases forming part of, the beta-sheet structure. Collectively, the 3 CDRs of a variable region determine its binding specificity. The exact boundaries of these CDRs have been defined according to different systems. The system described by Kabat (Kabat et al., SEQUENCES OF PROTEINS OF IMMUNOLOGICAL INTEREST (National Institutes of Health, Bethesda, Md. (1987) and (1991)) provides an unambiguous residue numbering system applicable to any variable region as well as precise residue boundaries defining the three CDRs (e.g., Kabat CDRs). Chothia et al. (Chothia and Lesk, 1987, J. Mol. Biol. 196: 901-17; Chothia et al., 1989, Nature 342: 877-83) found that certain sub-portions within Kabat CDRs adopt nearly identical peptide backbone conformations, despite having great diversity at the level of amino acid sequence. These sub-portions were designated as L1, L2, and L3 or H1, H2, and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan, 1995, FASEB J. 9: 133-39; MacCallum, 1996, J. Mol. Biol. 262(5): 732-45; and Lefranc, 2003, Dev. Comp. Immunol. 27: 55-77 (the IMGT definition). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding.

“Antibody fragments” comprise a portion of an intact antibody including the antigen binding regions. In some embodiments, an antibody fragment of the present disclosure is an antigen-binding fragment. Examples of antibody fragments include without limitation Fab, Fab′, F(ab)2, and Fv fragments; diabodies; linear antibodies; single-chain antibody molecules; and multispecific antibodies formed from antibody fragments.

A “monoclonal” antibody as used herein refers to an antibody obtained from a population of substantially homogeneous antibodies. For example, individual antibodies comprising the population may be identical except for possible mutations present in minor amounts. The modifier “monoclonal” indicates the character of the antibody as being obtained from a substantially homogeneous population of antibodies, and is not to be construed as requiring production of the antibody by any particular method.

### II. Methods

Certain aspects of the present disclosure relate to methods for producing a recombinant product (e.g., a polypeptide or multi-polypeptide complex, such as an antibody). In some embodiments, the methods comprise: (a) culturing a host cell in a culture medium in a first bioreactor (e.g., a production bioreactor of the present disclosure) under conditions suitable for expression of the polypeptide, wherein the first bioreactor is in fluid connection with an alternating tangential flow (ATF) microfilter such that the host cell, the culture medium, and the polypeptide from the first bioreactor contact the ATF microfilter; (b) transferring the polypeptide and a portion of the culture medium through the ATF microfilter into a second bioreactor (e.g., a harvest vessel of the present disclosure) that is in fluid connection with the ATF microfilter; (c) contacting the polypeptide and the portion of the culture medium in the second bioreactor with an ATF ultrafilter that is in fluid connection with the second bioreactor; and (d) collecting the polypeptide from the second bioreactor. In some embodiments, the methods comprise: (a) culturing a host cell in a culture medium in a first bioreactor (e.g., a production bioreactor of the present disclosure) under conditions suitable for expression of the polypeptide, wherein the first bioreactor is in fluid connection with an alternating tangential flow (ATF) microfilter such that the host cell, the culture medium, and the polypeptide from the first bioreactor contact the ATF microfilter; (b) filtering the polypeptide and a portion of the culture medium through the ATF microfilter into a second bioreactor (e.g., a harvest vessel of the present disclosure) that is in fluid connection with the ATF microfilter (e.g., such that the host cell is retained in the first bioreactor and the polypeptide and the portion of the culture medium pass into the second bioreactor); (c) filtering the portion of the culture medium in the second bioreactor through an ATF ultrafilter that is in fluid connection with the second bioreactor (e.g., such that the polypeptide is retained in the second bioreactor and culture medium is allowed to exit the second bioreactor); and (d) collecting the polypeptide from the second bioreactor. In some embodiments, the ATF microfilter causes the host cell to be retained in the first bioreactor and allows the polypeptide and the portion of the culture medium to pass into the second bioreactor. In some embodiments, the ATF ultrafilter causes the polypeptide to be retained in the second bioreactor and allows culture medium to exit the second bioreactor. Exemplary descriptions for systems and components thereof suitable for performing the methods of the present disclosure are provided herein. For example, suitable micro- and ultrafilters are described in section III infra. Any of the methods described herein may be performed using any of the systems of the present disclosure.

In some embodiments, the polypeptide is collected from the second bioreactor (e.g., a harvest vessel of the present disclosure) in one or more non-continuous batches. In some embodiments, the polypeptide is collected from the second bioreactor (e.g., a harvest vessel of the present disclosure) with lesser frequency than, or in fewer batches than, the polypeptide (and a portion of the culture medium) is transferred into the second bioreactor. For example, the polypeptide may be collected from the second bioreactor non-continuously (e.g., in one or more batches), while the polypeptide and cell culture medium are transferred into the second bioreactor and out of the first bioreactor (e.g., a production bioreactor of the present disclosure) via the ATF microfilter continuously.

In some embodiments, the host cell is cultured in the first bioreactor for a period of about 2 weeks to about 3 weeks, about 1 week to about 3 weeks, about 1 week to about 4 weeks, about 2 weeks to about 4 weeks, about 1 week to about 8 weeks, about 2 weeks to about 8 weeks, about 1 week to about 12 weeks, about 2 weeks to about 12 weeks, about 1 week to about 24 weeks, about 2 weeks to about 24 weeks, about 1 week to about 52 weeks, about 2 weeks to about 52 weeks, about 4 weeks to about 8 weeks, and the like. Without wishing to be bound to theory, it is thought that the host cell could potentially be cultured indefinitely in the first bioreactor (e.g., during continuous culturing) while the polypeptide is collected from the second bioreactor in non-continuous batches.

In some embodiments, the host cell is cultured in the first bioreactor for a period, and the polypeptide is collected from the second bioreactor in one batch per period. In some embodiments, the host cell is cultured in the first bioreactor for a period, and the polypeptide is collected from the second bioreactor in more than one batch per period. In some embodiments, the host cell is cultured in the first bioreactor for a period of about 2 weeks to about 3 weeks, about 2 weeks to about 4 weeks, about 2 weeks to about 8 weeks, up to about 8 weeks, up to about 12 weeks or longer than about 12 weeks, and the polypeptide is collected from the second bioreactor in one batch per period. In some embodiments, the host cell is cultured in the first bioreactor for a period of about 14 days to about 21 days, about 14 days to about 30 days, about 14 days to about 60 days, up to about 60 days, up to about 90 days or longer than about 90 days, and the polypeptide is collected from the second bioreactor in one batch per period. In some embodiments, the host cell is cultured in the first bioreactor for a period, and the polypeptide is collected from the second bioreactor in more than one batch per period. In some embodiments, the host cell is cultured in the first bioreactor for a period of more than about 3 weeks, more than about 4 weeks, more than about 8 weeks, more than about 12 weeks, or more than about 18 weeks, and the polypeptide is collected from the second bioreactor in more than one batch per period. In some embodiments, the host cell is cultured in the first bioreactor for a period of more than about 21 days, more than about 30 days, more than about 60 days, more than about 90 days, or more than about 120 days, and the polypeptide is collected from the second bioreactor in more than one batch per period. In some embodiments, the host cell is cultured in the first bioreactor for a period of about 2 weeks to about 3 weeks, about 1 week to about 3 weeks, about 1 week to about 4 weeks, about 2 weeks to about 4 weeks, about 1 week to about 8 weeks, about 2 weeks to about 8 weeks, about 1 week to about 12 weeks, about 2 weeks to about 12 weeks, about 1 week to about 24 weeks, about 2 weeks to about 24 weeks, about 1 week to about 52 weeks, about 2 weeks to about 52 weeks, or about 4 weeks to about 8 weeks; and the polypeptide is collected from the second bioreactor in 1, 2, 3, 4, 5, or more batches per period. In some embodiments, the host cell is cultured in the first bioreactor for a period of about 2 weeks to about 3 weeks, about 1 week to about 3 weeks, about 1 week to about 4 weeks, about 2 weeks to about 4 weeks, about 1 week to about 8 weeks, about 2 weeks to about 8 weeks, about 1 week to about 12 weeks, about 2 weeks to about 12 weeks, about 1 week to about 24 weeks, about 2 weeks to about 24 weeks, about 1 week to about 52 weeks, about 2 weeks to about 52 weeks, or about 4 weeks to about 8 weeks; and the polypeptide is collected from the second bioreactor in 1 batch per period. In some embodiments, the host cell is cultured continuously in the first bioreactor for a period of about 2 weeks to about 3 weeks, about 1 week to about 3 weeks, about 1 week to about 4 weeks, about 2 weeks to about 4 weeks, about 1 week to about 8 weeks, about 2 weeks to about 8 weeks, about 1 week to about 12 weeks, about 2 weeks to about 12 weeks, about 1 week to about 24 weeks, about 2 weeks to about 24 weeks, about 1 week to about 52 weeks, about 2 weeks to about 52 weeks, or about 4 weeks to about 8 weeks; and the polypeptide is collected non-continuously from the second bioreactor in 1, 2, 3, 4, 5, or more batches per period. In some embodiments, the host cell is cultured in the first bioreactor for a period of more than 2 weeks, more than 3 weeks, more than 4 weeks, more than 8 weeks, or more than 12 weeks, and the polypeptide is collected from the second bioreactor in more than one batch per period.

As demonstrated herein, the methods of the present disclosure are advantageous in that they allow for the concentration of the polypeptide in the second bioreactor (e.g., a harvest vessel of the present disclosure). In some embodiments, the frequency at which the polypeptide is collected from the second bioreactor (e.g., as described supra), and/or the number of batches used to collect the polypeptide from the second bioreactor, depends upon the desired concentration of the polypeptide (e.g., in the second bioreactor).

In some embodiments, the polypeptide is collected at a concentration of at least about 0.1 g/L, at least about 0.3 g/L, at least about 0.5 g/L, at least about 1 g/L, at least about 2 g/L, at least about 3 g/L, at least about 4 g/L, at least about 5 g/L, at least about 10 g/L, at least about 15 g/L, or at least about 20 g/L. In some embodiments, the polypeptide is collected at a concentration of at least about 1 g/L, at least about 5 g/L, at least about 7 g/L, at least about 8 g/L, or at least about 10 g/L. In some embodiments, the polypeptide is collected at a concentration of about 1 g/L to about 10 g/L, about 1 g/L to about 5 g/L, about 3 g/L to about 5 g/L, about 3 g/L to about 8 g/L, about 3 g/L to about 10 g/L, about 5 g/L to about 8 g/L, or about 5 g/L to about 10 g/L. In some embodiments, the polypeptide is collected at a concentration that is less than about any of the following concentrations (in g/L); 100, 90, 80, 70, 60, 50, 40, 30, 20, 15, 10, 9, 8, 7, 6, 5, 4, 3, 2, 1, 0.9, 0.8, 0.7, 0.6, 0.5, 0.4, 0.3, or 0.2. In some embodiments, the polypeptide is collected at a concentration that is greater than about any of the following concentrations (in g/L); 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, or 90. That is, the polypeptide can be collected at a concentration that is any of a range of concentrations having an upper limit of 100, 90, 80, 70, 60, 50, 40, 30, 20, 15, 10, 9, 8, 7, 6, 5, 4, 3, 2, 1, 0.9, 0.8, 0.7, 0.6, 0.5, 0.4, 0.3, or 0.2 g/L and an independently selected lower limit of 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, or 90 g/L, wherein the upper limit is greater than the lower limit.

In some embodiments, the host cell is cultured (e.g., in the first bioreactor) in a continuous manner, and the polypeptide is collected (e.g., from the second bioreactor) in a non-continuous manner. In some embodiments, the polypeptide and a portion of the culture medium are transferred into the second bioreactor (e.g., through the ATF microfilter) in a continuous manner, and the polypeptide is collected (e.g., from the second bioreactor) in a non-continuous manner. In some embodiments, the host cell is cultured (e.g., in the first bioreactor) in a continuous manner, the polypeptide and a portion of the culture medium are transferred into the second bioreactor (e.g., through the ATF microfilter) in a continuous manner, and the polypeptide is collected (e.g., from the second bioreactor) in a non-continuous manner.

In some embodiments, the host cell is cultured (e.g., in the first bioreactor) while the polypeptide and a portion of the culture medium are transferred into the second bioreactor (e.g., through the ATF microfilter) simultaneously. In some embodiments, the host cell is cultured (e.g., in the first bioreactor) more than once prior to contacting the polypeptide and the portion of the culture medium in the second bioreactor with the ATF ultrafilter and/or collecting the polypeptide (e.g., from the second bioreactor). In some embodiments, the host cell is cultured (e.g., in the first bioreactor) more than once prior to filtering a portion of the culture medium out of the second bioreactor via the ATF ultrafilter and/or collecting the polypeptide (e.g., from the second bioreactor). In some embodiments, the polypeptide and a portion of the culture medium are transferred into the second bioreactor (e.g., through the ATF microfilter) more than once prior to contacting the polypeptide and the portion of the culture medium in the second bioreactor with the ATF ultrafilter and/or collecting the polypeptide (e.g., from the second bioreactor). In some embodiments, the polypeptide and a portion of the culture medium are transferred into the second bioreactor (e.g., through the ATF microfilter) more than once prior to filtering a portion of the culture medium out of the second bioreactor via the ATF ultrafilter and/or collecting the polypeptide (e.g., from the second bioreactor). In some embodiments, the host cell is cultured (e.g., in the first bioreactor) and the polypeptide and a portion of the culture medium are transferred into the second bioreactor (e.g., through the ATF microfilter) both more than once prior to contacting the polypeptide and the portion of the culture medium in the second bioreactor with the ATF ultrafilter and/or collecting the polypeptide (e.g., from the second bioreactor). In some embodiments, the host cell is cultured (e.g., in the first bioreactor) and the polypeptide and a portion of the culture medium are transferred into the second bioreactor (e.g., through the ATF microfilter) both more than once prior to filtering a portion of the culture medium out of the second bioreactor via the ATF ultrafilter and/or collecting the polypeptide (e.g., from the second bioreactor). In some embodiments, the concentration of the polypeptide in the first bioreactor is kept constant, while the concentration of the polypeptide in the second bioreactor increases.

In some embodiments, the polypeptide and the portion of the culture medium in the second bioreactor are contacted with the ATF ultrafilter more than once prior to collecting the polypeptide. In some embodiments, a portion of the culture medium is filtered out of the second bioreactor via the ATF ultrafilter more than once prior to collecting the polypeptide. In some embodiments, the methods further comprise removing a second portion of the culture medium from the second bioreactor through the ATF ultrafilter, e.g., prior to collecting the polypeptide. In some embodiments, the second portion of the culture medium is less than the first portion. For example, in some embodiments, the second portion of the culture medium is removed from the second bioreactor when volume of culture medium in the second bioreactor reaches a predetermined volume. In some embodiments, the concentration of the polypeptide in the second bioreactor after removing the second portion is greater than concentration of the polypeptide in the second bioreactor prior to removing the second portion.

In some embodiments, the polypeptide is collected (e.g., from the second bioreactor) when the concentration of the polypeptide in the second bioreactor reaches a predetermined or threshold concentration. For example, in some embodiments, the polypeptide is collected (e.g., from the second bioreactor) when the concentration of the polypeptide in the second bioreactor reaches a concentration of at least about 0.1 g/L, at least about 0.3 g/L, at least about 0.5 g/L, at least about 1 g/L, at least about 2 g/L, at least about 3 g/L, at least about 4 g/L, at least about 5 g/L, at least about 8 g/L, at least about 10 g/L, at least about 15 g/L, or at least about 20 g/L. In some embodiments, the polypeptide is collected (e.g., from the second bioreactor) when the concentration of the polypeptide in the second bioreactor reaches a concentration of about 0.1 g/L, about 0.3 g/L, about 0.5 g/L, about 1 g/L, about 2 g/L, about 3 g/L, about 4 g/L, about 5 g/L, about 8 g/L, about 10 g/L, about 15 g/L, or about 20 g/L. In some embodiments, the polypeptide is collected (e.g., from the second bioreactor) when the concentration of the polypeptide in the second bioreactor reaches a concentration of about 1 g/L to about 10 g/L, about 1 g/L to about 5 g/L, about 1 g/L to about 8 g/L, about 3 g/L to about 5 g/L, about 3 g/L to about 8 g/L, about 3 g/L to about 10 g/L, about 5 g/L to about 8 g/L, or about 5 g/L to about 10 g/L.

In some embodiments, additional or fresh culture medium is introduced into the first bioreactor (e.g., during culturing of the host cell) prior to collecting the polypeptide. For example, additional culture medium can be introduced into the first bioreactor according to perfusion or fed-batch culturing techniques. In some embodiments, additional or fresh culture medium is introduced into the first bioreactor (e.g., during culturing of the host cell) at a rate that is approximately equivalent to a rate of transferring the portion of the culture medium (and polypeptide) from the first bioreactor into the second bioreactor (e.g., via the ATF microfilter). In some embodiments, the additional or fresh culture medium is the same as the culture medium used to culture the host cell in the first bioreactor. In some embodiments, the additional or fresh culture medium is different from the culture medium used to culture the host cell in the first bioreactor, such as a batch, fed-batch, or perfusion culture medium. For example, the culture can be supplemented with independent concentrated feeds of particular nutrients which may be difficult to formulate or are quickly depleted in cell cultures, including without limitation certain amino acids (e.g., cysteine/cystine, tyrosine, etc.), nutrients, etc.

In some embodiments, a host cell of the present disclosure is cultured (e.g., in a culture medium in a first bioreactor as described herein) in a perfusion cell culture. For example, in some embodiments, while the host cell is cultured in the first bioreactor (e.g., prior to collecting the polypeptide), additional or fresh culture medium is introduced into the first bioreactor, e.g., at a rate of about 1 volume of the first bioreactor per day. In some embodiments, while the host cell is cultured in the first bioreactor (e.g., prior to collecting the polypeptide), a portion of culture medium (including the polypeptide) is transferred from the first bioreactor to the second bioreactor (e.g., via the ATF microfilter) at a rate of about 1 volume of the first bioreactor per day. In some embodiments, while the host cell is cultured in the first bioreactor (e.g., prior to collecting the polypeptide), additional or fresh culture medium is introduced into the first bioreactor, e.g., at a rate of about 1 volume of the first bioreactor per day and a portion of culture medium (including the polypeptide) is transferred from the first bioreactor to the second bioreactor (e.g., via the ATF microfilter) at a rate of about 1 volume of the first bioreactor per day.

In some embodiments, e.g., after the polypeptide is collected, the methods further comprise purifying the collected polypeptide via one or more downstream purification processes. In some embodiments, the one or more downstream purification processes do not include depth filtration. In some embodiments, the one or more downstream purification processes comprise protein A affinity chromatography. In some embodiments, e.g., after the polypeptide is collected, the methods further comprise contacting the collected polypeptide with protein A.

Certain aspects of the present disclosure relate to culturing host cells in a culture medium, e.g., under conditions suitable for expression of a polypeptide (such as a recombinant polypeptide, like an antibody). A variety of suitable host cells, and methods for culturing such host cells, are known in the art. Typically the host cells are from a cell line that can be maintained in culture for an extended period of time and/or produce large amounts of a polypeptide product, such as a recombinant polypeptide. One or more polynucleotide(s) encoding the polypeptide (e.g., such as an expression vector, plasmid, etc.) can be introduced into and maintained in the host cell, e.g., via transformation, transfection, infection, or injection. Expression vectors contain the necessary elements for the transcription and translation of the inserted coding sequence, and optionally sequences that facilitate their replication, maintenance, and/or selection in the host cell. Methods which are known in the art can be used to construct expression vectors containing sequences encoding the produced proteins and polypeptides, as well as the appropriate transcriptional and translational control elements. These methods include in vitro recombinant DNA techniques, synthetic techniques, and in vivo genetic recombination. Such techniques are described in J. Sambrook et al., 2012, Molecular Cloning, A Laboratory Manual. 4^{th} edition Cold Spring Harbor Press, Plainview, N.Y.; F. M. Ausubel et al., 2013, Current Protocols in Molecular Biology, John Wiley & Sons, New York, N.Y.; Kaufman, R. J., Large Scale Mammalian Cell Culture, 1990; and the like.

In some embodiments, the host cell is a prokaryotic cell. In some embodiments, the host cell is a eukaryotic cell, such as a yeast, plant, or animal cell. In some embodiments, the host cell is a mammalian host cell or cell line. Suitable host cells are commercially available and/or available from the American Type Culture Collection (Manassas, Va.) and other depositories. Exemplary host cell types include, without limitation, MK2.7 cells, PER-C6 cells, Chinese hamster ovary cells (CHO), such as CHO-K1 (ATCC CCL-61), DG44 (Chasin et al., 1986, Som. Cell Molec. Genet., 12:555-556; Kolkekar et al., 1997, Biochemistry, 36:10901-10909; and WO 01/92337 A2), dihydrofolate reductase negative CHO cells (CHO/-DHFR, Urlaub and Chasin, 1980, Proc. Natl. Acad Sci. USA. 77:4216), and dp12.CHO cells (U.S. Pat. No. 5,721,121); monkey kidney cells (CV 1, ATCC CCL-70); monkey kidney CVI cells transformed by SV40 (COS cells, COS-7, ATCC CRL-1651); HEK 293 cells, myeloma cell lines such as Y0, NS0 and Sp2/0, 5L8 hybridoma cells, Daudi cells, EL4 cells, HeLa cells, HL-60 cells, K562 cells, Jurkat cells, THP-1 cells, Sp2/0 cells, baby hamster kidney cells (BHK, ATCC CCL-10); mouse sertoli cells (TM4, Mather, 1980, Biol. Reprod., 23:243-251); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); primary epithelial cells (e.g., keratinocytes, cervical epithelial cells, bronchial epithelial cells, tracheal epithelial cells, kidney epithelial cells and retinal epithelial cells) and established cell lines and their strains (e.g., human embryonic kidney cells (e.g., 293 cells, or 293 cells subcloned for growth in suspension culture, Graham et al., 1977, J. Gen. Virol., 36:59); TRI cells (Mather, 1982, Annals NY Acad Sci., 383:44-68); MCR 5 cells; FS4 cells; PER-C6 retinal cells, MDBK (NBL-1) cells, 911 cells, CRFK cells, MDCK cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA 13, 2RA cells, WISH cells, BS-C-I cells, LLC-MK2 cells, Clone M-3 cells, 1-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, and TH-I, B1 cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, M₁Cl₁ cells, CV-1 cells, COS-1 cells, COS-3 cells, COS-7 cells, African green monkey kidney cells (VERO-76, ATCC CRL-1587, VERO, ATCC CCL-81); DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDM₁C₃ cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, Swiss/3T3 cells, Indian muntac cells, SIRC cells, Cu cells, and Jensen cells, or derivatives thereof). For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Culture media suitable for culturing a variety of host cells are also known in the art and commercially available. Culture media can include media comprising serum as well as defined or serum-free media. In some embodiments, the culture medium is a perfusion culture medium. Examples of culture media known in the art include, without limitation, RPMI (e.g., RPMI 1640), Modified Dulbecco's Medium, Dulbecco's Modification of Eagle's Medium (DMEM) and variants thereof (e.g., with different amounts of glutamine, glucose, and the like), DME/F12, alpha MEM, Basal Medium Eagle with Earle's BSS, GMEM (Glasgow's MEM), GMEM with glutamine, Grace's Complete Insect Medium, Grace's Insect Medium, without FBS, Ham's F-10, with Glutamine, Ham's F-12, with Glutamine, IMDM with HEPES IP41 Insect Medium, 15 (Leibovitz)(2×), without Glutamine or Phenol Red, 15 (Leibovitz), without Glutamine, McCoy's 5A Modified Medium, Medium 199, MEM Eagle, without Glutamine or Phenol Red (2×), NCTC-109, with Glutamine, Richter's CM Medium, with Glutamine, RPMI 1640 with HEPES, Glutamine and/or Penicillin-Streptomycin, RPMI 1640, with Glutamine, RPMI 1640, without Glutamine. and Schneider's Insect Medium.

Conditions suitable for polypeptide expression (e.g., in a cell culture as described herein) are also known in the art and can be ascertained by one of skill in the art. Exemplary descriptions can be found, e.g., in J. Sambrook et al., 2012, Molecular Cloning, A Laboratory Manual, 4^{th} edition Cold Spring Harbor Press, Plainview, N.Y.; F. M. Ausubel et al., 2013, Current Protocols in Molecular Biology, John Wiley & Sons, New York, N.Y.; Kaufman, R. J., Large Scale Mammalian Cell Culture, 1990; Yazaki and Wu, Methods in Molecular Biology, Vol. 248 (B. K. C. Lo, ed., Humana Press, Totowa, N.J.), pp. 255-268 (2003); and the like.

The methods described herein may find use in the production of a wide range of polypeptide products (e.g., recombinant polypeptides). In some embodiments, the polypeptide is a secreted polypeptide, e.g., a polypeptide that is secreted into the culture medium by a host cell during culturing as described herein. In some embodiments, the polypeptide is an antibody (e.g., a monoclonal antibody) or antigen-binding fragment thereof. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. In some embodiments, the antibody is a monoclonal antibody, multispecific or bispecific antibody, single domain antibody, diabody, linear antibody, minibody, chimeric antibody, humanized antibody, human antibody, single chain or single arm antibody, or the like. Other examples of secreted polypeptides include, without limitation, enzymes, soluble T-cell receptors (TCRs), cytokines, interferons, growth factors, peptide hormones such as insulin, and derivatives thereof.

In some embodiments, the polypeptide is an antibody (e.g., a monoclonal antibody) or antigen-binding fragment thereof that binds an antigen. Exemplary antigens are provided below. Exemplary antibodies that bind the indicated antigen are shown in parentheses. It is contemplated that the methods described herein may be useful in the production of antibodies or antigen-binding fragments that bind any of the exemplary and non-limiting antigens described herein, e.g., infra.

In some embodiments, the antigen is a tumor-associated antigen. In some embodiments, the tumor-associated antigen is a transmembrane protein. For example, the following antigens are transmembrane proteins: ANTXR1, BAFF-R, CA9 (exemplary antibodies include girentuximab), CD147 (exemplary antibodies include gavilimomab and metuzumab), CD19, CD20 (exemplary antibodies include divozilimab and ibritumomab tiuxetan), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CLPTM1L, DPP4, EGFR, ERVMER34-1, FASL, FSHR, FZD5, FZD8, GUCY2C (exemplary antibodies include indusatumab), IFNAR1 (exemplary antibodies include faralimomab), IFNAR2, LMP2, MLANA, SIT1, TLR2/4/1 (exemplary antibodies include tomaralimab), TM4SF5, TMEM132A, TMEM40, UPK1B, VEGF, and VEFGR2 (exemplary antibodies include gentuximab).

In some embodiments, the tumor-associated antigen is a transmembrane transport protein. For example, the following antigens are transmembrane transport proteins: ASCT2 (exemplary antibodies include idactamab), MFSD13A, Mincle, NOX1, SLC10A2, SLC12A2, SLC17A2, SLC38A 1, SLC39A5, SLC39A6 also known as LIV1 (exemplary antibodies include ladiratuzumab), SLC44A4, SLC6A15, SLC6A6, SLC7A 11, and SLC7A5.

In some embodiments, the tumor-associated antigen is a transmembrane or membrane-associated glycoprotein. For example, the following antigens are transmembrane or membrane-associated glycoproteins: CA-125, CA19-9, CAMPATH-1 (exemplary antibodies include alemtuzumab), carcinoembryonic antigen (exemplary antibodies include arcitumomab, cergutuzumab, amunaleukin, and labetuzumab), CD112, CD155, CD24, CD247, CD37 (exemplary antibodies include lilotomab), CD38 (exemplary antibodies include felzartamab), CD3D, CD3E (exemplary antibodies include foralumab and teplizumab), CD3G, CD96, CDCP1, CDH17, CDH3, CDH6, CEACAM1, CEACAM6, CLDN1, CLDN16, CLDN18.1 (exemplary antibodies include zolbetuximab), CLDN18.2 (exemplary antibodies include zolbetuximab), CLDN19, CLDN2, CLEC12A (exemplary antibodies include tepoditamab), DPEP1, DPEP3, DSG2, endosialin (exemplary antibodies include ontuxizumab), ENPP1, EPCAM (exemplary antibodies include adecatumumab), FN, FN1, Gp100, GPA33, gpNMB (exemplary antibodies include glembatumumab), ICAM1, L1CAM, LAMP1, MELTF also known as CD228, NCAM1, Nectin-4 (exemplary antibodies include enfortumab), PDPN, PMSA, PROM1, PSCA, PSMA, Siglecs 1-16, SIRPa, SIRPg, TACSTD2, TAG-72, Tenascin, Tissue Factor also known as TF (exemplary antibodies include tisotumab), and ULBP1/2/3/4/5/6.

In some embodiments, the tumor-associated antigen is a transmembrane or membrane-associated receptor kinase. For example, the following antigens are transmembrane or membrane-associated receptor kinases: ALK, Axl (exemplary antibodies include tilvestamab), BMPR2, DCLK1, DDR1, EPHA receptors, EPHA2, ERBB2 also known as HER2 (exemplary antibodies include trastuzumab, bevacizumab, pertuzumab, and margetuximab), ERBB3, FLT3, PDGFR-B (exemplary antibodies include rinucumab), PTK7 (exemplary antibodies include cofetuzumab), RET, ROR1 (exemplary antibodies include cirmtuzumab), ROR2, ROS1, and Tie3.

In some embodiments, the tumor-associated antigen is a membrane-associated or membrane-localized protein. For example, the following antigens are membrane-associated or membrane-localized proteins: ALPP, ALPPL2, ANXA1, FOLR1 (exemplary antibodies include farletuzumab), IL13Ra2, IL1RAP (exemplary antibodies include nidanilimab), NT5E, OX40, Ras mutant, RGS5, RhoC, SLAMF7 (exemplary antibodies include elotuzumab), and VSIR.

In some embodiments, the tumor-associated antigen is a transmembrane G-protein coupled receptor (GPCR). For example, the following antigens are GPCRs: CALCR, CD97, GPR87, and KISS1R.

In some embodiments, the tumor-associated antigen is cell-surface-associated or a cell-surface receptor. For example, the following antigens are cell-surface-associated and/or cell-surface receptors: B7-DC, BCMA, CD137, CD 244, CD3 (exemplary antibodies include otelixizumab and visilizumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), FAS, FGFR1, FGFR2 (exemplary antibodies include aprutumab), FGFR3 (exemplary antibodies include vofatamab), FGFR4, GITR (exemplary antibodies include ragifilimab), Gpc3 (exemplary antibodies include ragifilimab), HAVCR2, HLA-E, HLA-F, HLA-G, LAG-3 (exemplary antibodies include encelimab), LY6G6D, LY9, MICA, MICB, MSLN, MUC1, MUC5AC, NY-ESO-1, OY-TES1, PVRIG, Sialyl-Thomsen-Nouveau Antigen, Sperm protein 17, TNFRSF12, and uPAR.

In some embodiments, the tumor-associated antigen is a chemokine receptor or cytokine receptor. For example, the following antigens are chemokine receptors or cytokine receptors: CD115 (exemplary antibodies include axatilimab, cabiralizumab, and emactuzumab), CD123, CXCR 4 (exemplary antibodies include ulocuplumab), IL-21R, and IL-5R (exemplary antibodies include benralizumab).

In some embodiments, the tumor-associated antigen is a co-stimulatory, surface-expressed protein. For example, the following antigens are co-stimulatory, surface-expressed proteins: B7-H3 (exemplary antibodies include enoblituzumab and omburtamab), B7-H4, B7-H6, and B7-H7.

In some embodiments, the tumor-associated antigen is a transcription factor or a DNA-binding protein. For example, the following antigens are transcription factors: ETV6-AML, MYCN, PAX3, PAX5, and WT1. The following protein is a DNA-binding protein: BORIS.

In some embodiments, the tumor-associated antigen is an integral membrane protein. For example, the following antigens are integral membrane proteins: SLITRK6 (exemplary antibodies include sirtratumab), UPK2, and UPK3B.

In some embodiments, the tumor-associated antigen is an integrin. For example, the following antigens are integrin antigens: alpha v beta 6, ITGAV (exemplary antibodies include abituzumab), ITGB6, and ITGB8.

In some embodiments, the tumor-associated antigen is a glycolipid. For example, the following are glycolipid antigens: FucGM1, GD2 (exemplary antibodies include dinutuximab), GD3 (exemplary antibodies include mitumomab), GloboH, GM2, and GM3 (exemplary antibodies include racotumomab).

In some embodiments, the tumor-associated antigen is a cell-surface hormone receptor. For example, the following antigens are cell-surface hormone receptors: AMHR2 and androgen receptor.

In some embodiments, the tumor-associated antigen is a transmembrane or membrane-associated protease. For example, the following antigens are transmembrane or membrane-associated proteases: ADAM12, ADAM9, TMPRSS11D, and metalloproteinase.

In some embodiments, the tumor-associated antigen is aberrantly expressed in individuals with cancer. For example, the following antigens may be aberrantly expressed in individuals with cancer: AFP, AGR2, AKAP-4, ARTN, BCR-ABL, C5 complement, CCNB1, CSPG4, CYP1B1, De2-7 EGFR, EGF, Fas-related antigen 1, FBP, G250, GAGE, HAS3, HPV E6 E7, hTERT, IDO1, LCK, Legumain, LYPDI, MAD-CT-1, MAD-CT-2, MAGEA3, MAGEA4, MAGEC2, MerTk, ML-IAP, NA 17, NY-BR-1, p53, p53 mutant, PAP, PLAVI, polysialic acid, PR1, PSA, Sarcoma translocation breakpoints, SART3, sLe, SSX2, Survivin, Tn, TRAIL, TRAIL1, TRP-2, and XAGE1.

In some embodiments, the antigen is an immune-cell-associated antigen. In some embodiments, the immune-cell-associated antigen is a transmembrane protein. For example, the following antigens are transmembrane proteins: BAFF-R, CD163, CD19, CD20 (exemplary antibodies include rituximab, ocrelizumab, divozilimab; ibritumomab tiuxetan), CD25 (exemplary antibodies include basiliximab), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CTLA4 (exemplary antibodies include ipilimumab), FASL, IFNAR1 (exemplary antibodies include faralimomab), IFNAR2, LAYN, LILRB2, LILRB4, PD-1 (exemplary antibodies include ipilimumab, nivolumab, pembrolizumab, balstilimab, budigalimab, geptanolimab, toripalimab, and pidilizumabsf), SIT1, and TLR2/4/1 (exemplary antibodies include tomaralimab).

In some embodiments, the immune-cell-associated antigen is a transmembrane transport protein. For example, Mincle is a transmembrane transport protein.

In some embodiments, the immune-cell-associated antigen is a transmembrane or membrane-associated glycoprotein. For example, the following antigens are transmembrane or membrane-associated glycoproteins: CD112, CD155, CD24, CD247, CD28, CD30L, CD37 (exemplary antibodies include lilotomab), CD38 (exemplary antibodies include felzartamab), CD3D, CD3E (exemplary antibodies include foralumab and teplizumab), CD3G, CD44, CLEC12A (exemplary antibodies include tepoditamab), DCIR, DCSIGN, Dectin 1, Dectin 2, ICAM1, LAMP1, Siglecs 1-16, SIRPa, SIRPg, and ULBP1/2/3/4/5/6.

In some embodiments, the immune-cell-associated antigen is a transmembrane or membrane-associated receptor kinase. For example, the following antigens are transmembrane or membrane-associated receptor kinases: Axl (exemplary antibodies include tilvestamab) and FLT3.

In some embodiments, the immune-cell-associated antigen is a membrane-associated or membrane-localized protein. For example, the following antigens are membrane-associated or membrane-localized proteins: CD83, IL1RAP (exemplary antibodies include nidanilimab), OX40, SLAMF7 (exemplary antibodies include elotuzumab), and VSIR.

In some embodiments, the immune-cell-associated antigen is a transmembrane G-protein coupled receptor (GPCR). For example, the following antigens are GPCRs: CCR4 (exemplary antibodies include mogamulizumab-kpkc), CCR8, and CD97.

In some embodiments, the immune-cell-associated antigen is cell-surface-associated or a cell-surface receptor. For example, the following antigens are cell-surface-associated and/or cell-surface receptors: B7-DC, BCMA, CD137, CD2 (exemplary antibodies include siplizumab), CD 244, CD27 (exemplary antibodies include varlilumab), CD278 (exemplary antibodies include feladilimab and vopratelimab), CD3 (exemplary antibodies include otelixizumab and visilizumab), CD40 (exemplary antibodies include dacetuzumab and lucatumumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), GITR (exemplary antibodies include ragifilimab), HAVCR2, HLA-DR, HLA-E, HLA-F, HLA-G, LAG-3 (exemplary antibodies include encelimab), MICA, MICB, MRC1, PVRIG, Sialyl-Thomsen-Nouveau Antigen, TIGIT (exemplary antibodies include etigilimab), Trem2, and uPAR.

In some embodiments, the immune-cell-associated antigen is a chemokine receptor or cytokine receptor. For example, the following antigens are chemokine receptors or cytokine receptors: CD115 (exemplary antibodies include axatilimab, cabiralizumab, and emactuzumab), CD123, CXCR4 (exemplary antibodies include ulocuplumab), IL-21R, and IL-5R (exemplary antibodies include benralizumab).

In some embodiments, the immune-cell-associated antigen is a co-stimulatory, surface-expressed protein. For example, the following antigens are co-stimulatory, surface-expressed proteins: B7-H 3 (exemplary antibodies include enoblituzumab and omburtamab), B7-H4, B7-H6, and B7-H7.

In some embodiments, the immune-cell-associated antigen is a peripheral membrane protein. For example, the following antigens are peripheral membrane proteins: B7-1 (exemplary antibodies include galiximab) and B7-2.

In some embodiments, the immune-cell-associated antigen is aberrantly expressed in individuals with cancer. For example, the following antigens may be aberrantly expressed in individuals with cancer: C5 complement, IDO1, LCK, MerTk, and Tyrol.

In some embodiments, the antigen is a stromal-cell-associated antigen. In some embodiments, the stromal-cell-associated antigens is a transmembrane or membrane-associated protein. For example, the following antigens are transmembrane or membrane-associated proteins: FAP (exemplary antibodies include sibrotuzumab), IFNAR1 (exemplary antibodies include faralimomab), and IFNAR2.

### III. Systems

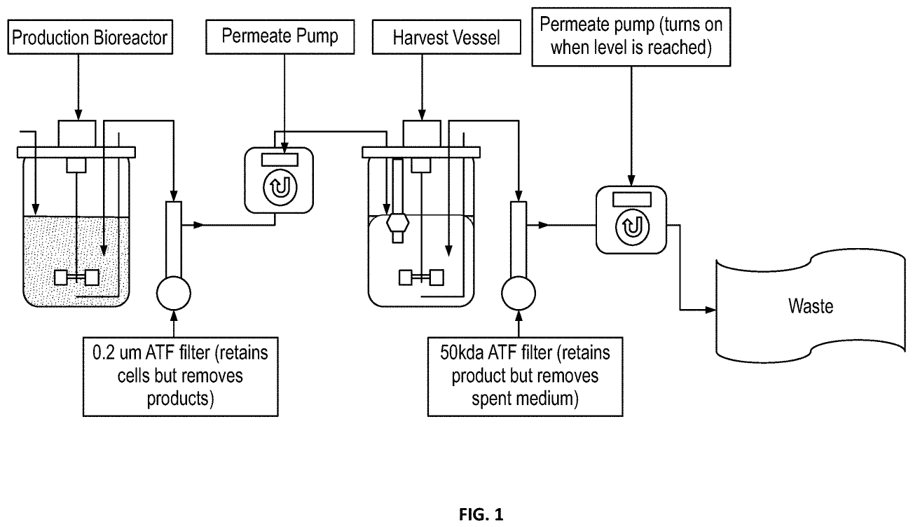

Certain aspects of the present disclosure relate to systems for producing a recombinant product (e.g., a polypeptide or multi-polypeptide complex, such as an antibody). Any of the systems described herein may find use in the any of the methods of the present disclosure. In some embodiments, the systems provide for batch production of the product, e.g., production in one or more non-continuous batches, which optionally can be purified via one or more downstream processes. In some embodiments, the systems comprise a first bioreactor (e.g., a production bioreactor of the present disclosure), an alternating tangential flow (ATF) microfilter, a second bioreactor (e.g., a harvest vessel of the present disclosure), and an ATF ultrafilter. In some embodiments, the first bioreactor is in fluid connection with the ATF microfilter, e.g., such that polypeptide and culture medium from the first bioreactor contact the ATF microfilter. In some embodiments, the ATF microfilter is in fluid connection with the first bioreactor and the second bioreactor. In some embodiments, the ATF microfilter causes cells to be retained in the first bioreactor and allows culture medium and the polypeptide to pass into the second bioreactor. In some embodiments, the second bioreactor is in fluid connection with the ATF microfilter and the ATF ultrafilter, e.g., such that polypeptide and culture medium from the first bioreactor are filtered through the ATF microfilter into the second bioreactor, and such that polypeptide and culture medium from the second bioreactor contact the ATF ultrafilter. In some embodiments, the ATF ultrafilter causes the polypeptide to be retained in the second bioreactor and allows culture medium to exit the second bioreactor. An exemplary and non-limiting system of the present disclosure is illustrated in FIG. 1.

In some embodiments, the first bioreactor is a stirred tank bioreactor. In some embodiments, the stirred tank bioreactor has a volume of about 3 L to about 3000 L. In some embodiments, the stirred tank bioreactor has a volume of about 3 L. In some embodiments, the first bioreactor is constructed using stainless steel or glass. In some embodiments, the first bioreactor uses single-use technology, e.g., comprising a material such as, without limitation, low density polyethylene (LDPE), linear low density polyethylene (LLDPE), or ultra-low density polyethylene (ULDPE). In some embodiments, the first bioreactor comprises one or more sensors. e.g., for pH, dissolved oxygen, temperature, and level. In some embodiments, one or more aspects of the contents of the first bioreactor (e.g., of the cell culture) are controlled using one or more sensors. In some embodiments, the one or more aspects include, without limitation, pH, dissolved oxygen, temperature, and/or level.

In some embodiments, the second bioreactor is a stirred tank bioreactor. In some embodiments, the stirred tank bioreactor has a volume of about 3 L to about 3000 L. In some embodiments, the stirred tank bioreactor has a volume of about 3 L. In some embodiments, the second bioreactor is constructed using stainless steel or glass. In some embodiments, the second bioreactor uses single-use technology, e.g., comprising a material such as, without limitation, low density polyethylene (LDPE), linear low density polyethylene (LLDPE), or ultra-low density polyethylene (ULDPE). In some embodiments, one or more aspects of the contents of the second bioreactor (e.g., of the polypeptide and culture medium) are controlled using one or more sensors. In some embodiments, the one or more aspects include, without limitation, pH, dissolved oxygen, temperature, and/or level.

In some embodiments, the ATF microfilter has a pore size sufficient to allow the polypeptide and culture medium through, while retaining the host cell in the first bioreactor. In some embodiments, the ATF microfilter has a pore size that is smaller than the host cell and larger than the polypeptide. For example, in some embodiments, the ATF microfilter has a pore size of about 750 kD to about 0.4 sum. In some embodiments, the ATF microfilter has a pore size of about 0.2 μm. In some embodiments, the ATF microfilter is constructed using a material comprising polyethersulfone (PES) or polysulfone (PS). In some embodiments, the filter assembly (e.g., of the ATF microfilter) comprises stainless steel. In some embodiments, the filter assembly (e.g., of the ATF microfilter) is reusable. In some embodiments, the filter assembly (e.g., of the ATF microfilter) is single-use.

In some embodiments, the ATF ultrafilter has a pore size or molecular weight cutoff sufficient to allow culture medium through, while retaining the polypeptide in the second bioreactor. In some embodiments, the ATF ultrafilter has a molecular weight cutoff that is less than a molecular weight of the polypeptide. In some embodiments, the ATF ultrafilter has a molecular weight cutoff of about 30 kD to about 100 kD or about 30 kD to about 50 kD. In some embodiments, the ATF ultrafilter is constructed using a material comprising polyethersulfone (PES) or polysulfone (PS). In some embodiments, the filter assembly (e.g., of the ATF ultrafilter) comprises stainless steel. In some embodiments, the filter assembly (e.g., of the ATF ultrafilter) is reusable. In some embodiments, the filter assembly (e.g., of the ATF ultrafilter) is single-use.

In some embodiments, the systems of the present disclosure further comprise a permeate pump, such as a perfusion, peristaltic, low shear, or double diaphragm pump. In some embodiments, the permeate pump is connected to the ATF microfilter and the second bioreactor and causes culture medium and the polypeptide to pass through the ATF microfilter into the second bioreactor. In some embodiments, the permeate pump is connected to the second bioreactor and the ATF ultrafilter and causes culture medium to exit the second bioreactor through the ATF ultrafilter. In some embodiments, the systems comprise 2 permeate pumps: a first permeate pump connected to the ATF microfilter and the second bioreactor that causes culture medium and the polypeptide to pass through the ATF microfilter into the second bioreactor; and a second permeate pump connected to the second bioreactor and the ATF ultrafilter that causes culture medium to exit the second bioreactor through the ATF ultrafilter.

In some embodiments, the permeate pump connected to the second bioreactor and the ATF ultrafilter (e.g., that causes culture medium to exit the second bioreactor through the ATF ultrafilter) is configured or programmed to operate when a predetermined volume (e.g., of polypeptide and culture medium) is reached in the second bioreactor. For example, the permeate pump can be configured or programmed to operate when a predetermined volume based in part on the total volume of the bioreactor is reached in the second bioreactor. In some embodiments, the permeate pump is configured or programmed to operate when the volume of polypeptide and culture medium in the second bioreactor is between 100 mL and 5000 L. In some embodiments, the permeate pump is configured or programmed to operate when the volume of polypeptide and culture medium in the second bioreactor is 1.5 L.

In some embodiments, the systems further comprise a waste outlet or waste collection vessel connected to the ATF ultrafilter. In some embodiments, the waste outlet or collection vessel is configured to remove or retain culture medium from the second bioreactor through the ATF ultrafilter.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Monoclonal Antibody (mAb) Concentration in a Harvest Vessel

Production and purification of monoclonal antibodies on a production scale is extremely valuable for both research and pharmaceutical production. However, process intensification through the use of perfusion at the production stage makes the purification process extremely difficult due to the need for a continuous harvest and purification. Another well-recognized challenge of the perfusion process is the need to clarify a massive quantity of cells.

This Example describes a methodology and system for concentrating and collecting polypeptide product (e.g., monoclonal antibody) in a harvest vessel separate from the production bioreactor that employs ATF micro- and ultrafilters. This allows the product to be concentrated (e.g., for a 2-3 week period, up to 60 days, or even longer) prior to any downstream purification (e.g., protein A chromatography). In addition, this method and system maintains one batch per production process, keeping downstream operations the same for traditional batch production (which is convenient for a multiproduct production facility), while also reducing costs (e.g., by eliminating the need for downstream depth filtration, centrifugation, or other methods to clarify large numbers of cells).

### Materials and Methods

Cell Line and Media

The cell line used for these experiments was an industry relevant Chinese Hamster Ovary (CHO) cell line expressing a recombinant monoclonal antibody (mAb). The basal media used for cell expansion and bioreactors was a chemically defined media for CHO cells. The cell line was thawed from a cryogenically preserved cell bank and scaled up in various sized single use shake flasks. Cells were expanded until the target volume was achieved to inoculate a 3 L stirred tank bioreactor with a 1.5 L working volume.

### Bioreactors

The first bioreactor stage was a cell mass generation stage (N−1) in the 3 L glass vessel with a 1.5 L working volume. A hollow fiber ATF perfusion filter was used during the N−1 stage to accelerate cell mass accumulation. The ATF filter used was made of polyethersulfone (PES) with a 0.2 micron pore size and a 1 mm lumen internal diameter. The device used to control the ATF perfusion filter was an ATF2 unit purchased from Repligen (Waltham, Mass.). The perfusion started on day 1 removing a target bioreactor volume of 0.25 volumes/day (375 mL) and this target increased by 0.25 volumes/day until a maximum of 1 volume/day was achieved (1.5 L). The level control was maintained at 1.5 L throughout culture through the use of a level float sensor that controlled the delivery of fresh basal media.

On day 7 of the culture batch the N−1 bioreactor transitioned to a production bioreactor where the permeate, containing mAb, line from the ATF filter was aseptically attached to the harvest vessel. The target bioreactor culture perfusion rate remained constant at 1 vessel volume/day removal. This permeate volume to the harvest vessel was maintained continuously. The fresh media composition changed on day 8 to include a complex nutrient feed. The ratio of basal media to the added nutrient feed was 85%:15%. The production bioreactor was maintained for a total of 16 days.

The harvest vessel was made up of a 3 L stirred glass bioreactor with a target volume controlled at 1.5 L. The only control used on the harvest vessel was agitation and level control, although it is contemplated that temperature and/or oxygen control could be used, e.g., in addition or alternative to agitation and/or level control, for product quality purposes. An ATF perfusion filter was attached to the harvest vessel and was controlled by an ATF2 unit purchased from Repligen (Waltham, Mass.). The ATF perfusion filter used was made of polyethersulfone (PES) with a 50 kDa pore size and a 1 mm lumen internal diameter. The perfusion rate of 1 vessel volume/day was maintained to match that of the continuous permeate flow from the production vessel. Consistent with the production vessel, the harvest vessel was maintained for 16 days.

### Process Analytics

Daily viable cell density (VCD) and cellular viability of the N−1 and N bioreactors was measured via a Vi-Cell Cell Counter (Beckman Coulter Life Sciences, Indianapolis, Ind.). Daily culture osmolality for the N−1 and N bioreactors was measured via 2020 Multi-sample micro osmometer (Advanced Instruments, Norwood, Mass.). Titer and metabolites were measured daily for all vessel types via a Cedex BioHT (Roche Diagnostics GmbH, Mannheim, Germany).

Results

A diagram of the system used is shown in FIG. 1. On day 8, collection of harvest material into the harvest tank from RX12 was started. The ATF ultrafilter on the harvest tank was able to withstand flux through the permeate pump; no breakthrough was observed based on titer measurements on the permeate line and waste bag.

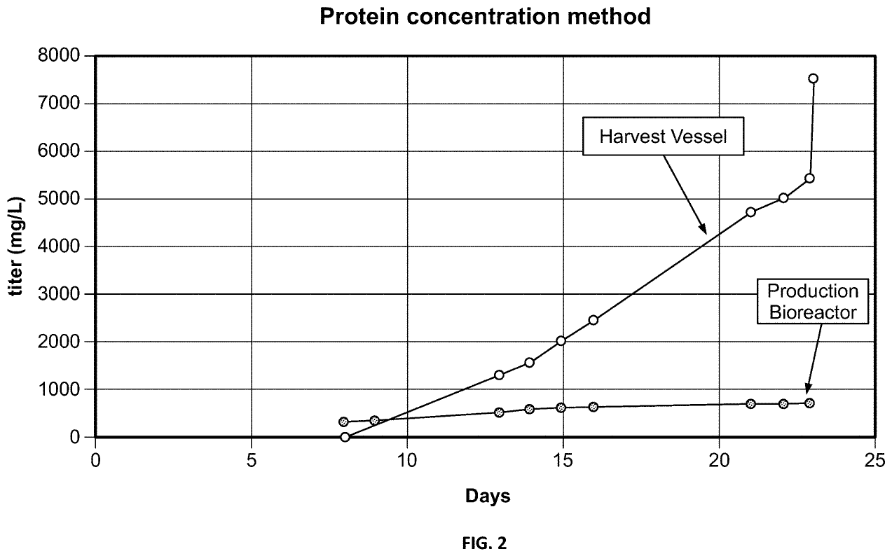

Titer in both the harvest tank and RX12 production bioreactor were measured over time (FIG. 2), demonstrating steady concentration of the mAb in the harvest tank with constant mAb concentration in the RX12. On the final day, mAb was concentrated by ⅓ in the harvest tank. Based on turbidity measurements, the ATF microfilter was able to reduce turbidity, as shown in Table 1.

TABLE 1
Turbidity of harvest fluids.

| Condition | turbidity (ntu) |
|---|---|
| cell culture | 553 |
| permeate line (rx12) | 1.2 |
| harvest tank | 10.6 |
| spun down sup | 79.1 |
| 0.22 um filtered sup | 69.3 |

The production cell culture accumulation peaked at a VCD of 30×10⁶ cell/mL with high cell viability throughout. The daily mAb titer levels in the production bioreactor ranged from 0.3-0.6 g/L (FIG. 2). The mAb concentration in the harvest vessel continued to increase throughout the process.

The most commonly used practice with process intensification to bridge the gap between cell culture and the first polypeptide capture stage is to utilize a break tank and continuous chromatography over an extended period of time (Konstantinov, K. B. and Cooney, C. L. (2015) J. Pharma. S. 104:P813-820). The challenges with this approach is in regards to quality and regulatory in that it becomes difficult to define a lot/batch of material in regards to specifications. The approach described in the above Example is innovative in that it retains all of the polypeptide produced in the second bioreactor by utilizing the ATF with an ultrafilter and maintains the downstream operations in a batch mode process, maintaining the single batch integrity for specification testing and release.

What is claimed is:

1. A method for producing a polypeptide, comprising:
(a) culturing, in a culture medium in a first bioreactor, a host cell that expresses the polypeptide under conditions suitable for expression of the polypeptide, wherein the first bioreactor is in fluid connection with an alternating tangential flow (ATF) microfilter such that the host cell, the culture medium, and the polypeptide from the first bioreactor contact the ATF microfilter;
(b) transferring the polypeptide and a portion of the culture medium through the ATF microfilter into a second bioreactor that is in fluid connection with the ATF microfilter, wherein the ATF microfilter causes the host cell to be retained in the first bioreactor and allows the polypeptide and the portion of the culture medium to pass into the second bioreactor;
(c) contacting the polypeptide and the portion of the culture medium in the second bioreactor with an ATF ultrafilter that is in fluid connection with the second bioreactor, wherein the ATF ultrafilter causes the polypeptide to be retained in the second bioreactor and allows culture medium to exit the second bioreactor; and
(d) collecting the polypeptide from the second bioreactor.

2. The method of claim 1, wherein the polypeptide is collected from the second bioreactor in one or more non-continuous batches.

3. The method of claim 2, wherein the host cell is cultured in the first bioreactor for a period of about 2 weeks to about 3 weeks, and wherein the polypeptide is collected from the second bioreactor in 1 batch per period.

4. The method of claim 2, wherein the host cell is cultured in the first bioreactor for a period of more than 3 weeks, and wherein the polypeptide is collected from the second bioreactor in more than one batch per period.

5. The method of any one of claims 1-4, wherein the polypeptide is collected at a concentration of at least about 1 g/L.

6. The method of claim 5, wherein the polypeptide is collected at a concentration of at least about 5 g/L.

7. The method of claim 5, wherein the polypeptide is collected at a concentration between about 5 g/L and about 8 g/L.

8. The method of any one of claims 1-7, wherein (a) and (b) are performed in a continuous manner, and wherein (d) is performed in a non-continuous manner.

9. The method of any one of claims 1-7, wherein (a) and (b) are performed simultaneously.

10. The method of any one of claims 1-9, wherein (a) and (b) are performed more than once prior to performing (c) and (d).

11. The method of any one of claims 1-10, wherein (c) is performed more than once prior to performing (d).

12. The method of any one of claims 1-11, further comprising, prior to (d), removing a second portion of the culture medium from the second bioreactor through the ATF ultrafilter.

13. The method of claim 12, wherein the second portion of the culture medium is less than the first portion.

14. The method of claim 12 or claim 13, wherein the second portion of the culture medium is removed from the second bioreactor when volume of culture medium in the second bioreactor reaches a predetermined volume.

15. The method of any one of claims 12-14, wherein concentration of the polypeptide in the second bioreactor after removing the second portion is greater than concentration of the polypeptide in the second bioreactor prior to removing the second portion.

16. The method of any one of claims 1-15, wherein (d) is performed when concentration of the polypeptide in the second bioreactor reaches a predetermined concentration.

17. The method of claim 16, wherein the predetermined concentration of the polypeptide is 5 g/L.

18. The method of any one of claims 1-17, further comprising, prior to (d) and during (a), introducing additional culture medium into the first bioreactor.

19. The method of claim 18, wherein additional culture medium is introduced into the first bioreactor at a rate that is approximately equivalent to a rate of transferring the portion of the culture medium from the first bioreactor into the second bioreactor in (b).

20. The method of any one of claims 1-17, wherein the host cell is cultured in a perfusion cell culture.

21. The method of claim 20, further comprising, prior to (d) and during (a), introducing additional culture medium into the first bioreactor at a rate of about 1 volume of the first bioreactor per day.

22. The method of claim 21, wherein, prior to (d), the portion of the culture medium is transferred from the first bioreactor to the second bioreactor in (b) at a rate of about 1 volume of the first bioreactor per day.

23. The method of any one of claims 1-22, wherein the polypeptide is a secreted polypeptide.

24. The method of any one of claims 1-23, wherein the polypeptide is a monoclonal antibody or antibody fragment.

25. The method of any one of claims 1-24, further comprising, after (d), purifying the collected polypeptide via one or more downstream purification processes.

26. The method of claim 25, wherein the one or more downstream purification processes do not include depth filtration.

27. The method of any one of claims 1-24, further comprising, after (d), contacting the collected polypeptide with protein A.

28. The method of any one of claims 1-27, wherein the ATF microfilter has a pore size of about 750 kD to about 0.4 μm.

29. The method of claim 28, wherein the ATF microfilter has a pore size of about 0.2 μm.

30. The method of any one of claims 1-29, wherein the ATF ultrafilter has a molecular weight cutoff of about 30 kD to about 100 kD.

31. The method of claim 30, wherein the ATF ultrafilter has a molecular weight cutoff of about 30 kD to about 50 kD.

32. The method of any one of claims 1-31, wherein the host cell is a mammalian host cell.

33. The method of claim 32, wherein the host cell is a Chinese hamster ovary (CHO) cell.

34. The method of any one of claims 1-33, wherein the culture medium is a defined culture medium.

35. A system for batch production of a polypeptide, comprising:
(a) a first bioreactor;
(b) an alternating tangential flow (ATF) microfilter;
(c) a second bioreactor; and
(d) an ATF ultrafilter;
wherein the first bioreactor is in fluid connection with the ATF microfilter;
wherein the ATF microfilter is in fluid connection with the first bioreactor and the second bioreactor, and wherein the ATF microfilter causes cells to be retained in the first bioreactor and allows culture medium and the polypeptide to pass into the second bioreactor;
wherein the second bioreactor is in fluid connection with the ATF microfilter and the ATF ultrafilter; and
wherein the ATF ultrafilter causes the polypeptide to be retained in the second bioreactor and allows culture medium to exit the second bioreactor.

36. The system of claim 35, wherein the first bioreactor is a stirred tank bioreactor.

37. The system of claim 36, wherein the first bioreactor is a stirred tank bioreactor with a volume of about 3 L to about 3000 L.

38. The system of claim 37, wherein the first bioreactor is a 3 L stirred tank bioreactor.

39. The system of any one of claims 35-38, wherein the second bioreactor is a stirred tank bioreactor.

40. The system of claim 39, wherein the second bioreactor is a stirred tank bioreactor with a volume of about 3 L to about 3000 L.

41. The system of claim 40, wherein the second bioreactor is a 3 L stirred tank bioreactor.

42. The system of any one of claims 35-41, further comprising a permeate pump connected to the ATF microfilter and the second bioreactor, wherein the permeate pump causes culture medium and the polypeptide to pass through the ATF microfilter into the second bioreactor.

43. The system of any one of claims 35-42, further comprising a second permeate pump connected to the second bioreactor and the ATF ultrafilter, wherein the second permeate pump causes culture medium to exit the second bioreactor through the ATF ultrafilter.

44. The system of claim 43, wherein the second permeate pump is configured to operate when a predetermined volume is reached in the second bioreactor.

45. The system of claim 44, wherein the predetermined volume is 1.5 L.

46. The system of any one of claims 35-45, wherein the ATF microfilter has a pore size of about 750 kD to about 0.4 μm

47. The system of claim 46, wherein the ATF microfilter has a pore size of about 0.2 μm.

48. The system of any one of claims 35-47, wherein the ATF ultrafilter has a molecular weight cutoff of about 30 kD to about 100 kD.

49. The system of claim 48, wherein the ATF ultrafilter has a molecular weight cutoff of about 30 kD to about 50 kD.

50. The system of any one of claims 35-49, further comprising a waste outlet or waste collection vessel connected to the ATF ultrafilter, wherein the waste outlet or collection vessel is configured to remove or retain culture medium from the second bioreactor through the ATF ultrafilter.
